(19) United States
(12) Patent Application Publication
(10) Pub. No.: US 2022/0034789 A1
(43) Pub. Date: Feb. 3, 2022

(54) NON-INVASIVE CONTINUOUS IN LINE ANTIFOULING OF ATR-MIR SPECTROSCOPIC SENSORS

(71) Applicant: Specshell ApS, Ballerup (DK)

(72) Inventors: Jens PILTOFT, Ballerup (DK); Christian PETERSEN, Ballerup (DK); Erik HOFFMAN-PETERSEN, Ballerup (DK); Andreas KUNOV-KRUSE, Ballerup (DK)

(73) Assignee: Specshell ApS, Ballerup (DK)

(21) Appl. No.: 17/276,445

(22) PCT Filed: Oct. 29, 2019
(86) PCT No.: PCT/DK2019/050325

§ 371 Date: Mar. 15, 2021

(30) Foreign Application Priority Data
Oct. 30, 2018 (DK) PA201870702

(51) Int. Cl. G01N 21/15; G01N 21/552; G02B 27/00; A61L 2/10

(52) U.S. Cl. CPC G01N 21/15; G01N 21/552; A61L 2202/11; A61L 2/10; G01N 2201/0638; G02B 27/0006

(57) ABSTRACT

An attenuated total reflectance mid-infra-red crystal antifouling method for preventing or removing biofilm from an ATR-MIR crystal is disclosed herein.

The present invention relates to improvement of the performance and enhancement of the application of Attenuated Total Reflectance-Mid Infrared (ATR-MIR) spectrometers in industrial process monitoring.

More specifically a method to prevent biofilm formation on the attenuated total reflectance mid-infra-red crystal is disclosed herein.

### BACKGROUND

This project has received funding from the European Union's Horizon 2020 research and innovation programme under grant agreement No 880138.

The citation and incorporation of patent documents herein is done for convenience only and does not reflect any view of the validity, patentability, and/or enforceability of such patent documents.

When measuring Mid infrared (MIR) spectres using the Attenuated Total Reflectance (ATR) MIR spectroscopy technique, generation of biofilms on the surface of the ATR-MIR crystal can result in misleading signals or completely erroneous signals not reflecting contributions from the sample, but instead reflecting signals from the biofilm.

In some cases the substrate of the monitored process is a carbohydrate conversion, e.g. an ethanol or lactic acid fermentation or an enzymatic conversion of starch containing crops or glucose isomerization. In such cases, faulty measurements from an in-line/at-line ATR-MIR spectroscopic probe would be especially critical, as the spectra recorded of biofilms would have a strong similarity to the spectra of the substrate. This would of cause be extremely confusing for the chemometric calibrations and algorithms applied to analyzing spectral data from the ATR-MIR spectrometer. In addition, there is a large chance that the error might not even be discovered and wrong and costly process control decisions could be made based on the faulty ATR-MIR spectroscopic sensor data.

Even a thin or partial biofilm coverage of the ATR crystal can cause serious problems. The concern of biofilm blockage of the ATR crystal is a reason why ATR-MIR often is considered unsuitable for analysis of industrial processes as biofilm formation could require intensive cleaning efforts to keep the ATR crystal clean as described in e.g. WO 2009/121423 A1 (PCT/EP2008/057380).

### DESCRIPTION OF THE INVENTION

Disclosed herein is an ATR-MIR (attenuated total reflectance mid-infra-red) crystal antifouling method for cleaning an ATR-MIR crystal so that biofouling on the ATR-MIR crystal is avoided. The ATR-MIR crystal is positioned in an ATR-MIR unit used for measuring ATR-MIR spectra of a sample.

Throughout the patent, antifouling will refer to means to prevent biofilm formation on the ATR-MIR crystal and/or cleaning the ATR-MIR crystal in case of a biofilm formation.

The ATR-MIR crystal comprises a sample surface side in direct contact with the sample, and an MIR light surface side onto which MIR light is directed allowing the MIR light to penetrate through the ATR-MIR crystal and interact with the sample trough an evanescent wave, where the MIR light is reflected from the sample surface side of the ATR-MIR crystal after interaction with the sample on the sample surface side.

The ATR-MIR crystal antifouling method comprises the step of illuminating the sample surface side of the ATR-MIR crystal with ultra violet radiation transmitted through the MIR light surface side of the ATR-MIR crystal originating from an ultra violet (UV) radiation source emitting ultra-violet (UV) light, whereby bacteria in samples in contact with the ATR-MIR crystal are killed, thereby preventing biofilms from being created on the ATR-MIR crystal.

The ATR-MIR crystal may in one or more embodiments be positioned in an ATR-MIR plate.

The above ATR-MIR crystal antifouling method enables measurements of MIR spectra repeatedly using ATR-MIR spectroscopy in environments that allow the growth of microorganisms. This will allow ATR-MIR to be applied successfully as a process monitoring tool in a range of industries where the technique presently is not used due to potential interference with biofilms.

### BRIEF DESCRIPTION OF THE DRAWINGS

FIGS. 1A-C show different examples of ATR-MIR crystals and the MIR lights pathway in the setup.

FIGS. 2A-C show the formation of a biofilm on an ATR-MIR crystal.

FIGS. 3A-D show the formation of a biofilm on an ATR-MIR crystal and the effect on a sample being in contact with the ATR-MIR crystal.

FIGS. 4A-J show different combinations of ATR-MIR crystals and one or more ultra violet sources.

FIGS. 5A-D show different versions of ATR-MIR units with an ATR-MIR crystal and an ultra violet source.

FIG. 6 shows a schematic illustration of the critical angle to obtain a total reflection on the interface.

FIG. 7 shows the amount of light lost in a starch slurries when a wavelength of 250 nm was radiating through a 1 cm quartz cuvette.

FIG. 8 shows a simple monochromatic UV spectroscopic setup comprising a 267 nm UV LED and a detector, each with a connector for a quartz fiber, used to investigate the interaction between bacteria and UV evanescent waves.

FIG. 9 shows a bioreactor vessel fitted with linear array ATR-MIR spectroscopic analyzer and a UV LED and quartz lens.

FIG. 10 A-B show different combinations of an ATR-MIR spectroscopic analyzer used for bioreactor vessel monitoring (A) or inline monitoring (B). The ATR-MIR spectroscopic analyzer is fitted with a small quartz fiber connecter to an UV LED.

FIG. 11 shows a plot from the calibration of the COD calibration.

### DESCRIPTION OF PREFERRED EMBODIMENTS

All headings and sub-headings are used herein for convenience only and should not be construed as limiting the invention in any way.

The description herein of any aspect or embodiment of the invention using terms such as “comprising”, “having,” “including,” or “containing” with reference to an element or elements is intended to provide support for a similar aspect or embodiment of the invention that “consists of”, “consists essentially of”, or “substantially comprises” that particular element or elements, unless otherwise stated or clearly contradicted by context, e.g. a composition described herein as comprising a particular element should be understood as also describing a composition consisting of that element, unless otherwise stated or clearly contradicted by context. It will be further understood that the terms “comprises,” “comprising,” “includes” and/or “including,” when used in this specification, specify the presence of stated features, integers, steps, operations, elements, and/or components, but do not preclude the presence or addition of one or more other features, integers, steps, operations, elements, components, and/or groups thereof.

Unless otherwise defined, all terms used herein (including technical and scientific terms) have the same meaning as commonly understood by those skilled in the art to which this invention pertains. It will be further understood that terms, such as those defined in commonly used dictionaries, should be interpreted as having a meaning that is consistent with their meaning in the context of the relevant art and will not be interpreted in an idealized or overly formal sense unless expressly so defined in the present specification.

As used herein, the singular forms “a,” “an,” and “the” are intended to include the plural forms, including “at least one,” unless the content clearly indicates otherwise. “At least one” is not to be construed as limiting “a” or “an.”

The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Various examples are described hereinafter with reference to the figures. It should also be noted that the figures are only intended to facilitate the description of the examples. They are not intended as an exhaustive description of the claimed invention or as a limitation on the scope of the claimed invention. In addition, an illustrated example needs not have all the aspects or advantages shown. An aspect or an advantage described in conjunction with a particular example is not necessarily limited to that example and can be practiced in any other examples even if not so illustrated, or if not so explicitly described.

When describing the embodiments, the combinations and permutations of all possible embodiments have not been explicitly described. Nevertheless, the mere fact that certain measures are recited in mutually different dependent claims or described in different embodiments does not indicate that a combination of these measures cannot be used to advantage. The present invention envisage all possible combinations and permutations of the described embodiments.

### MIR Spectroscopy

MIR instruments operate in the MIR region, which is typically from 400 up to 4000 cm⁻¹ (wavelength from 2500 nm up to 25000 nm). The absorptions of MIR photons brings the molecular bond in the irradiated sample to vibrate either through bending or stretching deformations. Each vibration type corresponds to the absorption of infrared light peaking around one specific wavelength. The sum of all these vibrations and the corresponding absorptions of MIR photons at the respective wavelength result in a MIR spectra.

The position and intensity of each photon absorption and the corresponding vibration obey the rules of quantum mechanics, and can therefore be predicted and theoreticized using different approximations and models. Due to these mechanisms behind the MIR spectroscopy, no vibration in practice exceeds 4000 cm⁻¹, and the majority of interesting spectra lies well below 3700 cm⁻¹.

The true MIR spectrometers are often confused with Near IR (NIR) spectrometers, which despite the similar name are very different. NIR spectrometers operates above the MIR wavenumbers, typically up to around 10.000 cm⁻¹, or in some spectrometers all the way up to visible or even ultraviolet (UV) wavelengths. None of the primary “true” vibrations are found in the NIR spectra, only overtones and combination bands of the actuals vibrations can be seen in NIR spectra. The overtones are typically scrambled and contain much less analytical and chemometric value.

While MIR spectroscopy is able to give very rapid and detailed insight of a sample, the sampling is in the basic spectrometer form much more cumbersome. This is a significant disadvantage compared to UV-VIS-NIR spectrometric methods where a simple “long path” cuvette can be used as sample holder or a relatively simple reflection probe. The absorption in the MIR region is usually very strong requiring the sample to be diluted in a suspension, either using an inert mineral oil such as Nujol or a MIR transparent such as potassium bromide. The use of MIR on neat liquid samples has traditionally required very “low path” cuvette systems, reducing thickness typically to around 10-100 μm. Especially for aqueous samples the path length should preferable be very little to ensure transparency, as water has strong absorptions in the MIR region. These very thin cuvettes are impractical when applied to analysis samples from biological and biochemical processes that are often in the form of heterogeneous slurries, which will need to be filtered prior to injection to the narrow cuvette system.

### Total Reflection and its Use in ATR-MIR Spectroscopy

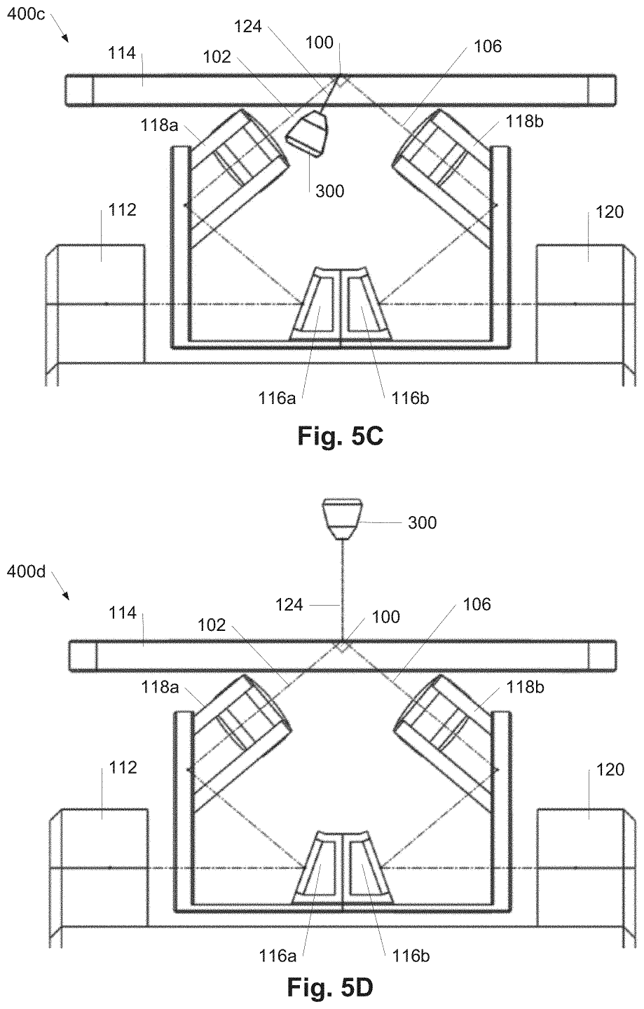

When light travels from a high refractive index crystal towards a lower refractive index material, like in FIG. 6 with angle defined from the perpendicular to the interface between the two materials, two different scenarios are possible: If the light enters in an angle lower than the so called critical angle (with respect to the interface perpendicular), some of the light beam will be reflected and some will enter the lower refractive index material—this is illustrated in FIG. 6 right hand side. However, if the angle is larger than the critical angle the beam will perform a total reflection on the interface. The critical angle is derived from Snell's formula (Eq. 0):

θ_{critical}=sin⁻¹(n₂/n₁) Eq. 0

where n₁ is the refractive index of the crystal and n₂ is the refractive index of the material in contact with the crystal.

From the point of total reflection an evanescent wave will propagate into the lower refractive index material. The depth of penetration of the evanescent wave into the lower refractive index material is given in Eq. 1

$${d}_{p} = \frac{λ}{2 {π ( {n}_{1}^{2} {sin}^{2} θ - {n}_{2}^{2} )}^{1 / 2}} \qquad Eq . 1$$

where λ is the wavelength of the light, θ is the angle of incidence relative to a perpendicular of the surface of the crystal and n₁ and n₂ is the crystal and sample refractive indices respectively.

This is exploited in a sampling MIR technique, which has become gradually more popular the last decades, known as Attenuated Total Reflectance (ATR) MIR spectroscopy. This exploit the so called attenuated total reflectance that happens when a wave of light is reflected on the interface of two materials with a significant difference in refractive index (in the case of ATR-MIR this is a high refractive crystal and a lower refractive index sample in direct contact with the crystal). The evanescent wave has a suitable interaction with the samples to obtain high quality IR spectra. Typically, the penetration depth of the evanescent wave is in the order of 1 μm in the MIR region (according equation 1). This is conveniently low for most MIR spectroscopic purposes ensuring full transparency in the whole MIR range (4000-400 cm⁻¹), even in highly absorbing samples. In the UV region the evanescent wave will be much shorter, typically in the order from tens to a hundred nano meters (10 to 100 nm).

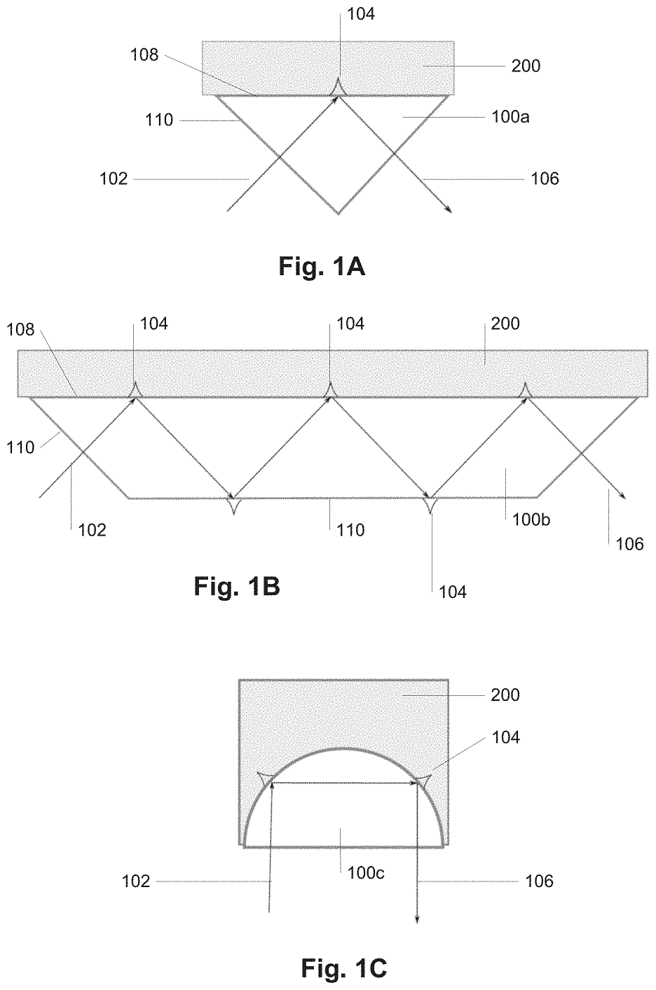

Different schematic and non-limiting examples are shown on FIGS. 1A-C showing ATR-MIR crystals 100a, 100b, 100c, MIR light illuminating 102 the ATR-MIR crystal, evanescent waves 104, MIR light reflected back 106 from the ATR-MIR crystal and the sample 200. Note that the size of the evanescent wave symbol 104 is much larger than the actual evanescent wave, for clarity.

In a crystal setup where the MIR beam is reflected a single time at the crystal-sample interface as shown in FIG. 1A, the effective path length is equal to dₚ from equation 1. In some cases several reflections are advantageous and the crystal geometry can be designed in several ways to achieve the MIR beam to be reflected several times on the crystal-sample interface as shown in FIGS. 1B and 1C. This gives a larger effective path length (EPL), which can be an advantage when dealing with weaker absorbing samples resulting in a better signal to noise ratio (SNR).

EPL=N·dₚ Eq. 2

Where, N is the amount of reflections on the interface between the crystal and the sample, and dₚ is the depth of penetration calculated from Eq.1. However, it is very important to stress that increasing the amounts of reflections does not increase the actual penetration dept. As an example: a 10 reflection ATR crystal where the evanescent wave penetrates 1 μm into the sample at a certain wavenumber, will have an accumulative EPL of 10 μm. The increased EPL will enhance the signal to noise ratio significantly. However, the depth of penetration is still just 1 μm at each reflection point. Hence, sample species that are not within 1 μm of the crystal interface will not contribute to the recorded spectra.

An often beneficial side effect of the low ATR penetration depth is, that particles significantly larger than the depth of penetration will only contribute negligible to the recorded spectra, thus only the liquid part of the slurry will be recorded. As the EPL is constant ATR-MIR spectroscopy obey Lambert Beers law:

A=ε·c·l=ε·c·EPL Eq. 3

where A is the absorbance, ε0 is the extinction coefficient, c is the concentration, and l is the effective sample path length equal to EPL.

Due to the suitable and fixed depth of penetration, straightforward application, and no need for filtering, ATR-MIR is an attractive choice for continuous in situ at-line/in-line monitoring of chemical, biochemical and biological processes in a broad range of industries. ATR-MIR spectroscopy can thus be used to give precise real time insight in the composition of the whole reactor or pipeline allowing better process control.

The sampling can be achieved in various different ways, just as long as the crystal surface is in contact with the reactor fluid at all times of analysis as described in WO 2015/155353 A1 (PCT/EP2015/057887). Some non-limiting examples of continuous sampling concepts:
1. Sample fluid is continuously extracted from the reactor or pipeline, e.g. through a small tube and an external pump, or the pressure inside the reactor. Then it is led over an ATR-MIR spectrometer adjacent to the reactor equipped with a flow-through attachment. The fluid sample can either be discarded or cycled back to the reactor or pipeline.
2. A probe with comprising ATR-MIR crystal is inserted directly into the reactor or pipeline, and the MIR beam to and from the probe is led by fibres, conduits, waveguide, or similar.
3. A compact ATR-MIR spectrometer is installed directly on the side of the reactor or pipeline. A small hole in the reactor/pipeline wall allows the sample to flow across the crystal, allowing for continuous in line monitoring of the process.

### Blockage of ATR-Crystal by Biofilms

The micrometer scale of the evanescent MIR wave is excellent for obtaining a correct statistical representation of chemical and biochemical species in solutions as the scale of the molecules are in the size range of sub nanometer to around tens or a hundred nanometers for very large molecules like proteins. However the continuous at-line/in-line process monitoring is especially vulnerable to blockage of the ATR-crystal as the technique is completely dependent on that the crystal surface being exposed to a representative sample of the whole reactor/pipeline at all times.

ATR crystal blockage due to particles are of less concern; larger particles do not contribute significantly to the spectra, while deposits of fine particulate impurities on the ATR crystal can in most cases be counteracted simply by ensuring a sufficient flow over the crystal, and a clever design and placement of flow-through or immersion probes used.

The formation of biofilms, however, pose significant challenge for the use of continuous ATR-MIR in chemical, biochemical, and biological processes.

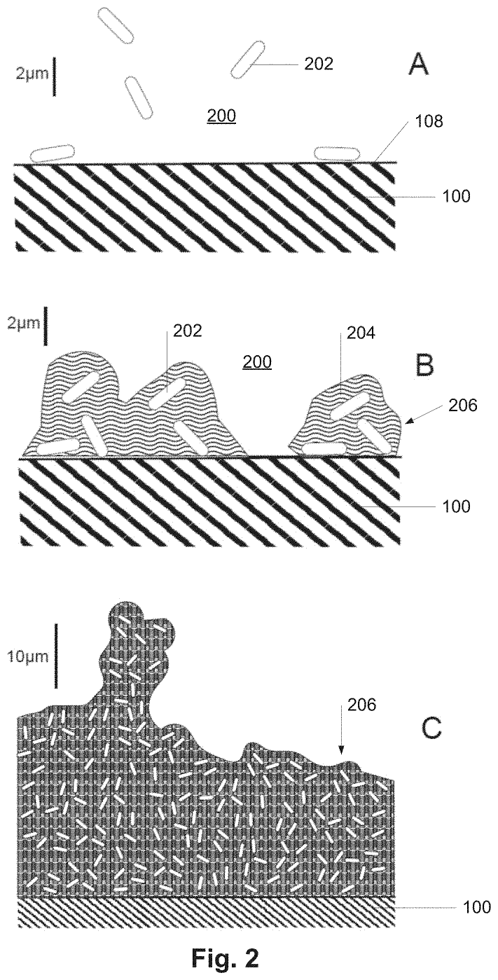

Biofilm can be formed by most microorganisms, like e.g. bacteria or algae. Prior to biofilm formation, the free floating (planktonic) microorganisms move around in a solution more or less randomly according to Brownian motions or by simple gliding. In the initial step some planktonic microorganisms attach to the surface, at first very weak by van der Waals and electrostatic forces. In FIG. 2A an initial attachment is shown, where only a few microorganisms 202 in the sample 200 are attached to the sample surface side 108 of the ATR-MIR crystal 100.

In the next step the microorganisms 202 starts to make a stronger irreversible attachment to the sample surface side 108 by production of an extracellular polymeric substance (EPS) matrix 204 as seen in FIG. 2B. The composition of the EPS is primarily insoluble polysaccharides, some proteins, and in addition to attaching the film to the sample surface side 108 of the ATR-MIR crystal 100, the EPS 204 also serves as a protective slimy matrix for the microorganisms making them very resistant to both chemicals, antibiotics and heat. This first phase of the biofilm formation is called the conditioning phase, and can under optimal conditions be completed in a few hours. Rough surfaces, particulates and old biological material, serves as promoting factors in the conditioning phase.

After the conditioning phase, the biofilm typically enters a fast exponential growth phase until it reaches a steady thickness of several hundred micrometers, or even millimeters or centimeters as shown in FIG. 2C. The biofilm 206 constantly spreads though releasing microbes into the surrounding environment. Obviously, unwanted biofilm causes a series of problems in industry, if present in significant amounts in parts of or the entire process, but will not be further elaborated on in the present disclosure.

Unfortunately, biofilm also causes problems if present at or around the ATR-MIR crystal. Even the slightest biofilm formation, like in the first phase of the biofilm formation, will cause severe problems for the ATR-MIR sensor long before they start to be problematic in the overall process.

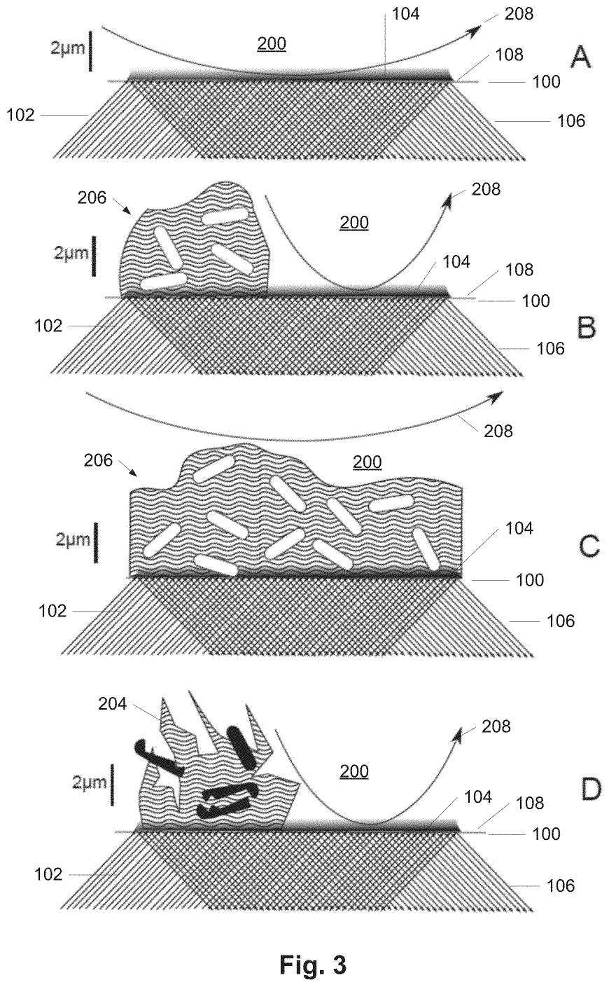

FIG. 3 compares different scenarios caused by biofilm contamination of the ATR crystal. FIG. 3A shows a section of a clean ATR crystal 100 with the sample surface side 108 facing a reactor containing the sample 200. FIG. 3A can be seen as an optimal working ATR-MIR probe in an industrial process. The small straight arrows show the flux of the incoming MIR beam 102/the reflected MIR light 106 and the gradient grey area represent the evanescent wave 104. The large curved arrow 208 represents the fluid flow over the crystal 100, which is ensuring a representative sample 200 from the process is in contact with the ATR-MIR crystal 100 at all time.

FIG. 3B shows a scenario where the sample surface side 108 of the ATR-MIR crystal 100 is partly covered with a biofilm 206. In this scenario the MIR spectra will represent an unknown mixture of the EPS matrix substrate 204, the bacteria and the sample still flowing over the uncovered part of the ATR-MIR crystal 100.

FIG. 3C shows the scenario where the ATR-MIR crystal is completely covered with a biofilm 206. The ATR-MIR spectra recorded in this scenario now exclusively contains signals from the content of the EPS matrix 204, and the flow from the actual analyte 208 is completely blocked and controlled by the diffusion properties of the EPS matrix 204. It is worth noticing that this happens independently of the thickness of the biofilm 206, and even a microscopic layer invisible to the naked eye can be enough to cause separation between the process fluid and the ATR-MIR crystal 100.

FIG. 3D shows an incomplete removal of a biofilm 206 during a cleaning in place (CIP) cycle or a heating between two process batches. In this case, all the microbes are killed by a disinfectant/CIP procedure but part of the sticky EPS matrix 204 is still attached to the sample surface side 108 of the ATR-MIR crystal 100. This example shows that even microscopic and biologically inactive remains of a biofilm 206 are able to cause failures in the ATR-MIR measurement as the spectra recorded still contains signals from an unknown amount of the EPS matrix 204.

All of the above scenarios are very likely to occur even in well-sanitized process equipment for several reasons, which will not be further disclosed in here. However, often biofilm formation at process equipment surfaces is prevented by special coatings, either very smooth, or containing germicidal chemical species. Unfortunately, due to the inherently low penetration of the evanescent MIR wave, a preventive coating of the MIR crystal surface may be unsatisfiable.

This invention relates to a method for ensuring a clean ATR crystal surface in industrial processes, over long periods of operation, where biofilms are known to form on surfaces of process equipment, such as e.g. pipeline and reactor walls, with as little as possible interference with the process.

Disclosed herein is therefore more specifically an ATR-MIR crystal antifouling method for cleaning an ATR-MIR crystal so that biofouling on the ATR-MIR crystal is avoided, the ATR-MIR crystal being positioned in an ATR-MIR unit used for measuring ATR-MIR spectra of a sample, wherein the ATR-MIR crystal comprises a sample surface side in direct contact with the sample, and an MIR light surface side onto which MIR light is directed allowing the MIR light to penetrate through the ATR-MIR crystal and interact with the sample trough an evanescent wave, where the MIR light, after interacting with the sample, is reflected from the MIR surface side of the ATR-MIR crystal, wherein the ATR-MIR crystal antifouling method comprises the step of illuminating the sample surface side of the ATR-MIR crystal with radiation from an ultra violet radiation source emitting ultra-violet light, whereby bacteria in samples in contact with the ATR-MIR crystal are killed, thereby preventing biofilms from being created on the ATR-MIR crystal; and wherein UV radiation is directed at the ATR-MIR crystal from the MIR light surface side, thereby passing through the ATR-MIR crystal before illuminating the sample surface side of the ATR-MIR crystal.

In one or more embodiments of the invention, the ATR-MIR crystal is facing a reactor or unit containing the sample. The sample will normally be a liquid or slurry.

In one or more embodiments, the ultra violet radiation source is selected from an UVC generating lamp such as a UV-light emitting diode, a UV laser, a mercury lamp, a deuterium lamp, a cold cathode lamp, or combinations hereof.

In one or more embodiments, the UV radiation source is a broad spectrum lamp emitting a very high percentage of light from the UVC spectral range between 100-300 nm.

In one or more embodiments, the UV radiation source is a UV Laser, or a UV light emitting diode (LED) emitting a UV light, peaking around a narrow wavelength band, chosen somewhere in the 240-280 nm range. High content of UVC in this range is one of the preferred embodiments due to the high germicidal effect caused by specific absorption in DNA molecules.

In one or more embodiments, the UV radiation source is a lamp emitting a high proportion UVB radiation between 280-315 nm.

In one or more embodiments, the UV radiation source is chosen from lamps with a peak emissions in the visible region between 400-750 nm, however emitting a significant proportion of UVA and UVB light.

In one or more embodiments, the UV radiation source is a high wattage visible/UVA LED or laser device emitting light between 350-750 nm. In an example, the peak emission is chosen from the blue-violet region from 350-500 nm.

In one or more embodiments, several UV radiation sources, such as at least two, such as at least three, such as at least four, are used simultaneously, wherein the UV radiation sources are directed at the ATR-MIR crystal from the MIR light surface from different angles.

High energy UV destroys microbe DNA preventing that the microbes divide further. The UV radiation source illuminating the ATR-crystal surface will kill microbes attaching to sample surface side of the ATR-MIR crystal before they produce any EPS. Without any EPS matrix around the dead microbes, they are easily removed even by a gentle fluid flow or eventually by Brownian motions.

If necessary the protective action of an UV radiation source can be combined with physical means to remove any biofilm formed on the surface such as ultrasound, a high pressure water jet stream, heat, or an automated brush, scraper, wiper, or similar.

In one or more embodiments, ultra sound is used in combination with radiation from the ultra violet radiation source.

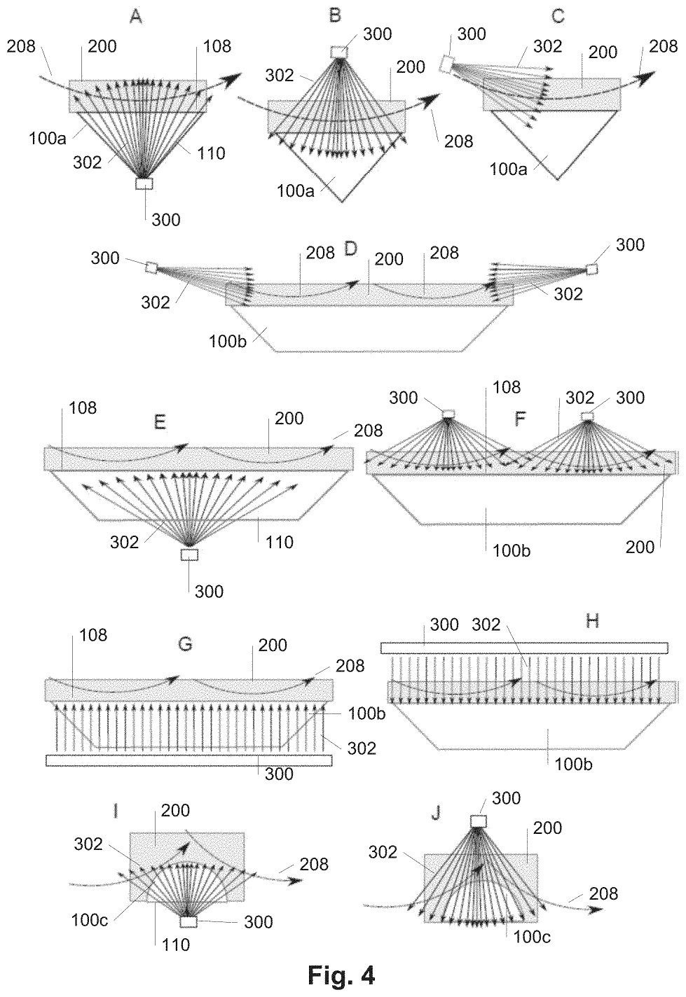

In one embodiment of the invention shown in FIG. 4A, the UV radiation source 300 is placed under a single reflection ATR-crystal 100a with the direction of the UV radiation beam 302. The UV radiation source is placed under the ATR-crystal (100, 100a, 100b, 100c) with the direction of a UV radiation beam lower than the critical angle of the ATR-MIR crystal. The angle needs to be lower than the critical angle or else the beam will perform a total reflection on the interface. The ATR-MIR crystal 100a is transparent at the wavelength of the radiation emitted by the UV radiation source 300, thus the ultra violet radiation is transmitted through the MIR-ATR crystal from the MIR light surface side 110 of the ATR-MIR crystal 100 to the sample surface side 108 of the ATR-MIR crystal 100 facing the sample 200.

It is important that the incident angle of the UV radiation beam 302 is lower than the critical angle, preferably not too far from being perpendicular in relation to the sample surface side 108 of the ATR-MIR crystal 100a, in order to avoid total reflection of the UV radiation beam 302. A benefit of the embodiment shown in FIG. 4A is that the UV radiation source 300 is placed outside the reactor/pipeline system, but inside an ATR-MIR spectroscopic probe.

FIG. 4E shows a similar variation of the embodiment shown in FIG. 4A, where an elongated transparent trapezoid ATR crystal 100b is used instead of the triangular crystal 100a. In FIG. 4E, the UV radiation source 300 is again placed under the ATR-MIR crystal 100b in an angle lower than the critical angle in relation to the sample surface side 108 of the ATR-MIR crystal 100b. Again this allow the ATR-MIR crystal 100b to transmit the ultra violet radiation to the sample surface side 108 of the ATR-MIR crystal 100b.

In one or more embodiments, the UV radiation beam has a shape matching the shape of the sample surface side 108 of the ATR-MIR crystal. Hereby a uniform radiation of the entire sample surface side is ensured.

In another similar embodiment shown in FIG. 4G, the UV radiation source 300 has an elongated tube-like shape matching the length of the trapezoid ATR-crystal 100b.

In yet another similar embodiment shown in FIG. 4I, the UV radiation source 300 illuminates a rounded ATR-MIR crystal 100c from the MIR light surface side 110 of the ATR-MIR crystal 100c.

In one or more embodiments, the MIR spectrometer is constructed using various techniques within the field such as a FTIR spectrometer using a Michelson interferometer; such as a FTIR spectrometer using a Sagnac interferometer, such as using a linear array combined with linear variable filter, such as using a single wavelength pyroelectric chip for detection of one wavelength, such as using infrared up conversion principle, or such as using a synchrotron generated IR beam, or combinations hereof.

In one or more embodiments, an optic fiber is used to direct the UV radiation beam from the UV radiation source to the ATR-MIR crystal.

In another embodiment one or more UV radiation sources 300 are placed in close proximity to an ATR-MIR crystal 100 and the radiation 302 is aimed perpendicular to the sample surface side 108 of the ATR-IR crystal 100 thereby passing through the ATR-MIR crystal before illuminating the sample surface side 108 of the ATR-MIR crystal 100.

Other examples of possible illumination not part of the present invention is exemplified in FIGS. 4B, 4F and 4J, where the FIG. 4B shows an embodiment with one UV radiation source 300 and a triangular ATR-MIR crystal 100a, FIG. 4F shows an embodiment with two UV radiation sources 300 and an elongated transparent trapezoid ATR crystal 100b, and FIG. 4J shows an embodiment with two closely positioned UV radiation sources 300 and a rounded ATR-MIR crystal 100c. One or more UV radiation sources 300 are placed in close proximity to an ATR-MIR crystal 100 and the radiation 302 is aimed perpendicular to the sample surface side 108 of the ATR-IR crystal 100 thereby passing through the sample 200 before illuminating the sample surface side 108 of the ATR-MIR crystal 100. Due to the short distance between the UV radiation source 300 and the ATR-MIR crystal 100, the UV radiation beam 302 is able to irradiate the sample surface side 108 of the ATR-MIR crystal 100 even though the beam 302 might travel through sample 200.

Another exemplification are shown in FIG. 4H, where the UV radiation source 300 has an elongated tube-like shape matching the length of the trapezoid ATR-crystal 100b with the radiation 302 aimed directly towards the sample surface side 108 of the ATR-IR crystal 100b.

In another example not part of the present invention, one or more UV radiation sources 300 are placed in close proximity to the ATR-MIR crystal 100 at a shallow angle between the UV radiation beam 302 and the sample surface side 108 of the ATR-MIR crystal 100. The angle should be low to allow the UV radiation beam 302 to irradiate the sample surface side 108 of the ATR-MIR crystal 100, while limiting the intensity of light entering the crystal. Examples of such as setup is shown in FIGS. 4C and 4D, where FIG. 4C shows a triangular ATR-MIR crystal 100a and one UV radiation source 300, and FIG. 4D shows an elongated transparent trapezoid ATR crystal 100b and two UV radiation sources 300.

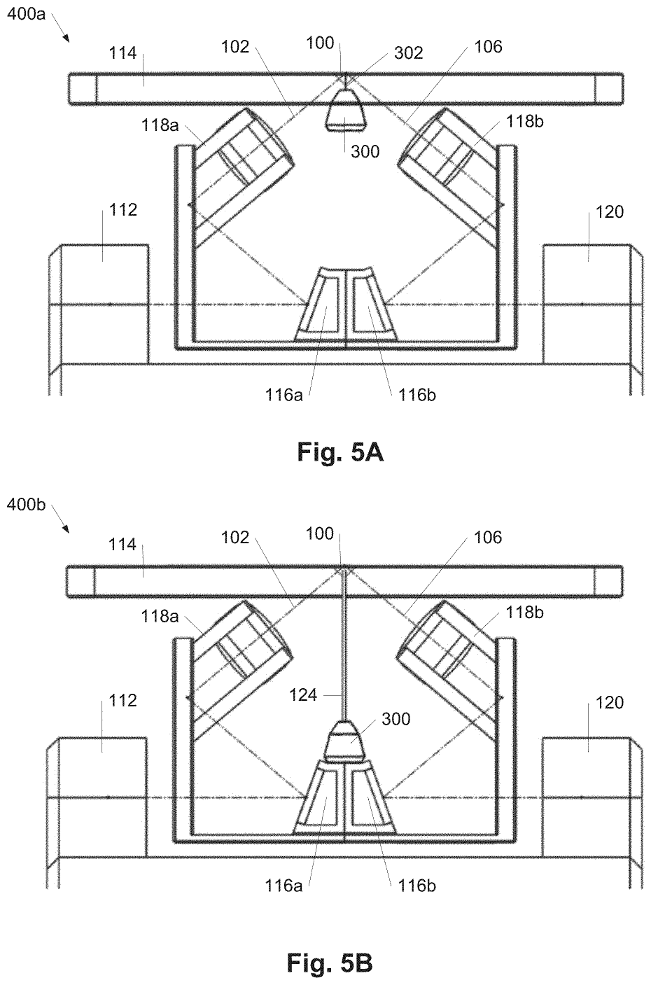

An optical design showing an ATR-MIR spectroscopic analyzer 400a is illustrated schematically in FIG. 5A. The ATR-MIR spectroscopic analyzer 400a comprises an MIR emitter 112 emitting MIR light 102, where the MIR light 102 is directed at the MIR light surface side by means of a mirror 116a and other optics 118a. Likewise, the MIR light reflected back 106 from the ATR-MIR crystal 100 is directed to an MIR detector 120 by means of a mirror 116a and other optics 118b. The ATR-MIR crystal 100 is placed in an ATR plate 114. The UV radiation source 300 is directing an UV radiation beam 302 at the MIR light surface side 110 of the ATR-MIR crystal 100. The result is that the UV radiation beam 302 is aimed directly towards the ATR-MIR crystal, perpendicular to the sample surface side of the ATR-MIR crystal 100 and penetrating the ATR-MIR crystal 100 from the MIR light surface side 110 of the ATR-MIR crystal 100. The continuous illumination of the diamond surface with UVC light prevents the formation of biofilms.

FIG. 5B shows an alternative embodiment of an ATR-MIR spectroscopic analyzer 400b shown in FIG. 5A, where the prevention of the biofilm formation is obtained using an optical fibre 124 to direct the UV radiation beam 302 from the UV radiation source 300 to the MIR light surface side 110 of ATR-MIR crystal 100.

FIG. 5C shows an alternative embodiment of an ATR-MIR spectroscopic analyzer 400c compared to the ones shown in FIGS. 5A and 5B. In FIG. 5C, the UV radiation source 300 is aimed at an angle so that the UV radiation beam 302 pass through the MIR light surface side 110 to the MIR sample surface side 108 while limiting the intensity of light dispersion inside the ATR-MIR spectroscopic analyzer 400c.

FIG. 5D shows an alternative example of an ATR-MIR spectroscopic analyzer 400d not part of the present invention, where the UV radiation source 300 is positioned such that the UV radiation beam is directed at the sample surface side 108 of the ATR-IR crystal 100 by means of a fiber 124.

The examples of ATR-MIR spectroscopic analyzers 400 shown in FIGS. 5A-C are only non-limiting examples, as ATR-MIR spectroscopic analyzers can be built in many other ways.

In one or more embodiments, the method further comprising the steps of continuously measuring ATR-MIR spectra of the sample in real time at wavelengths between 400-3500 cm⁻¹ and directing ultra violet radiation at the ATR-MIR crystal:
at least part of the time during the measurement of the ATR-MIR spectra, or
all the time during the measurement of the ATR-MIR spectra, or
in between measurements of some of the ATR-MIR spectra, or
in between measurements of all of the ATR-MIR spectra.

In one or more embodiments, the method further comprising the steps of continuously measuring ATR-MIR spectra of the sample in real time at wavelengths between 400-3500 cm⁻¹ and periodically directing ultra violet (UV) radiation at the ATR-MIR crystal for a first preset time period.

In one or more embodiments, the method further comprising the steps of continuously measuring ATR-MIR spectra of the sample in real time at wavelengths between 400-3500 cm⁻¹ and periodically not directing ultra violet (UV) radiation at the ATR-MIR crystal for a second preset time period.

In one or more embodiments, the method further comprising the steps of continuously measuring ATR-MIR spectra of the sample in real time at wavelengths between 400-3500 cm⁻¹, periodically directing ultra violet (UV) radiation at the ATR-MIR crystal for a first preset time period, and periodically not directing ultra violet (UV) radiation at the ATR-MIR crystal for a second preset time period.

In one or more embodiments, the first preset time period is between 0.001 seconds and 21600 seconds, such as between 0.01 seconds and 18000 seconds, such as between 0.1 seconds and 14400 seconds, such as between 1 second and 10800 seconds, such as between 10 seconds and 7200 seconds, such as between 30 seconds and 3600 seconds, such as between 30 seconds and 1800 seconds, such as between 0.001 seconds and 60 seconds.

In one or more embodiments, the second preset time period is between 0.001 seconds and 24 hours, such as between 1 second and 24 hours, such as between 1 minute and 24 hours, such as between 1 hour and 24 hours, such as between 1 hour and 12 hours, such as between 1 hour and 6 hours, such as between 1 minute and 6 hours, such as between 1 second and 6 hours, such as between 0.001 seconds and 6 hours.

In one or more embodiments, the sample is an aqueous slurry or solution comprising naturally occurring carbohydrates and proteins, such as e.g. sucrose, starch and other carbohydrates, crops and residues such as e.g. barley, wheat, rye, oat, corn, rice, potatoes, straw, wood, corn stover, sugar cane, bagasse, or others.

In one or more embodiments, the ATR-MIR unit measures ATR-MIR spectra of an enzymatic or microbial conversion process such as a mashing process conducted prior to fermentation of beer or an ethanol fermentation process.

This invention includes all modifications and equivalents of the subject matter recited in the claims and/or aspects appended hereto as permitted by applicable law.

The present invention is further illustrated by the following examples, which are not to be construed as limiting the scope of protection. The features disclosed in the foregoing description and in the following examples may, both separately or in any combination thereof, be material for realizing the invention in diverse forms thereof.

### Example 1—Buildup of Biofilm in a Brewery

An ATR-MIR spectroscopic analyzer such as the one shown in 400a is constructed to monitor mashing of malted barley in a 10⁵ Liter scale brewery. The analyzer is build using a robust custom build flow-through attachment, on top of a modified Specac Golden Gate Diamond ATR-Cell, together with a small FTIR spectrometer. The analyzer 400a is connected to the 10⁵ Liter unit in a continuous sampling system using ¾ inch silicone and steel tubing and an industrial peristaltic pump. The sampling is performed on the unfiltered slurry at a high flow rate of 3 L/min.

Three mashing batches are run every day with no production during night hours.

A CIP cycle on mashing unit is run once every week, cleaning out all inner equipment surfaces including the analyzer. However, especially in periods where ATR-MIR spectra are not recorded, the analyzer's diamond ATR crystal is in some cases covered with biofilm, making the subsequent analytical results from the ATR-MIR spectroscopic analyzer process unreliable.

### Example 2—Application to Ethanol Fermentation

In a fermenter unit, a fiber optic ATR-MIR spectrometer is used for monitoring the ethanol production from various different carbohydrate sources. The monitoring is done in a sideline to the main fermenter unit where an ATR-MIR immersion probe is inserted. The probe end is a stainless steel tube containing IR optics and a diamond ATR-MIR crystal at the end. Some combinations of carbohydrate substrate and commercial yeast strains are known to form biofilm on the fermenter walls. In these cases the EPS of the biofilm would block the fermentation slurry to reach the evanescent wave, and cause faulty chemometric predictions.

### Example 3—Penetration of UV Light in a Slurry (Comparative Example)

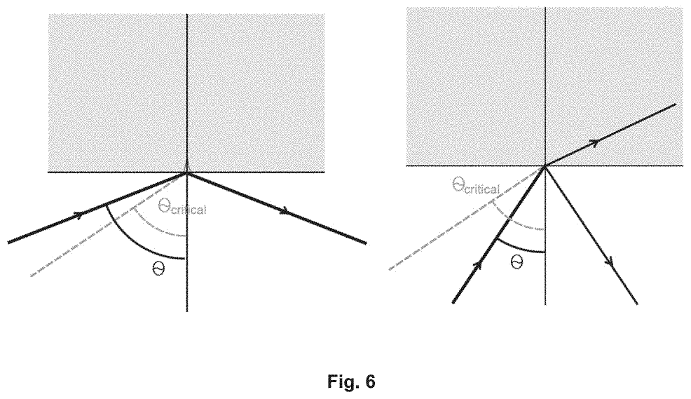

The amount of light lost in a starch slurries were investigated. Slurries with different dry matter concentrations were fabricated. A wavelength of 250 nm was used to investigate the amount of lost UV light when radiating through a 1 cm quartz cuvette. The results are shown in FIG. 7 Error! Reference source not found. The amount of light loss due to absorption and scattering was very significant even at the lowest concentration investigated. Even at 0.97 w/w % starch, 99.4% of the UV light is, absorbed and/or scattered by the particles. This shows that a light configurations coming from the samples side are not suited to deliver a biocidal dose of UV light to the ATR crystal sample surface side in a slurry mixture. This situation is illustrated in FIGS. 4. B, C, D, F, H, and J, and are all unsuited for continuous and robust inline antifouling of ATR crystals in process fluids. It further shows that a robust germicidal effect in slurries will not be achieved in practice from by UV irradiation from the sample side.

### Example 4—Evanescent UV Wave has No Germicidal Effect

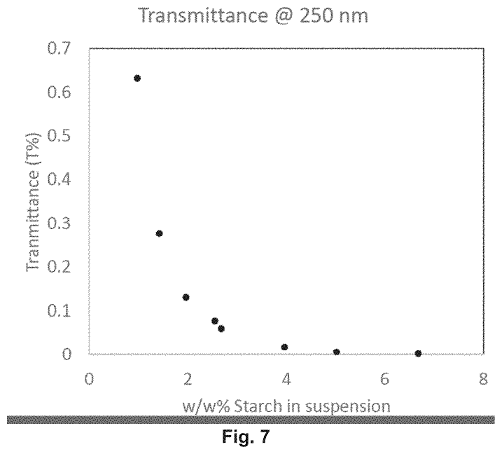

The interaction with bacteria and UV evanescent waves was investigated using a simple monochromatic UV spectroscopic setup comprising a 267 nm UV LED 500 and a detector 510, each with a fiber coupler 504 for a quartz fiber 506. Detector 510 and UV LED 500 each was connected to each end of a quartz U-bend probe 508 through quartz fibers 506. The quartz U-bend probe 508 was made out of a hundred micron diameter naked quarts fiber and was placed inside a bioreactor vessel 512. A schematic of the setup is shown in FIG. 8. To test the sensitivity and linearity of the quartz U-bend probe 508, a solutions of L-Tryptophan was measured in the bioreactor vessel 512. Tryptophan was chosen as a test molecule as it has a broad absorption peak at around 275 nm very close to the broad absorption peak of DNA peaking around 260 nm. Tryptophan showed linear response (absorbance) even at lower concentrations below 100 mg/L with a detection limit at around 20 mg/L. This demonstrates that the evanescent waves originating all the way around the quartz U-bend probe 508 have good contact with the solution in the bioreactor vessel 512, as the many reflections and evanescent waves generates a suitable large Effective path length (EPL). Using eq. 1 the depth of penetration is estimated be around 60 nm at.

A procedure was developed to enable a biofilm of Pseudomonas putida to form on the bioreactor vessel's 512 walls and the quartz U-bend probe 508 surface. After this the UV light was turned on. However, no absorbance from the DNA in the bacteria could be detected. Then the quartz U-ben probe 508 and bioreactor vessel 512 were both cleaned and sterilized. Attempts to prevent film formation on the quartz U-bend probe 508 was tested under same growth conditions with the UV light on. The film formed regardless of the UV light turned on or off. After this it was concluded that a germicidal effect preventing biofilm formation could not be achieved from the evanescent waves alone. Even though the total sum for evanescent waves have good interaction with the liquid in contact with the quartz U-bend probe 508, i.e. a significant EPL, each evanescent wave only propagates around 60 nm outside the quartz U-bend probe 508. This means that each evanescent wave will have poor, if any, contact with bacteria surrounding the quartz U-bend probe 508, which have a size from around thousand to several thousand nano meters. This demonstrates the UV light needs to be refracted out in the sample side to have a germicidal effect thus it must be transmitted in an angle lower than the critical angle with respect to the surface perpendicular (see FIG. 6)

### Example 5—UV Light Applied Close to Perpendicularly

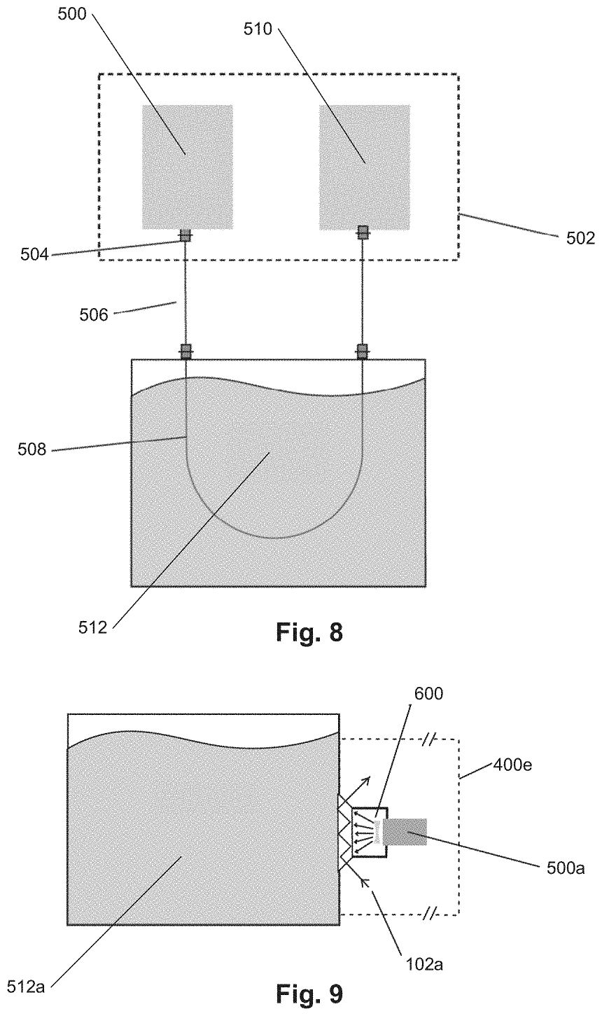

In a bioreactor vessel 512a a linear array ATR-MIR spectroscopic analyzer 400e with a triple reflection sapphire ATR crystal, is mounted directly on the side of an industrial fermenter, similar to the setup in FIG. 9. During some fermentation, biofilm formation was observed on the fermenter walls and the ATR crystal. While causing no problems for the overall fermentation process, the biofilm formation forming on the ATR crystal would block a representative amount of the fermentation broth to reach the evanescent wave of the reactor. Thus, a small UV LED 500a and a quartz lens 600 was installed right under the sapphire crystal, allowing a sufficient amount of UV light to be transmitted through the sapphire and into the fermenter broth creating a small germicidal field just around the crystal surface in contact with the broth. The UV radiation was chosen of such a low effect so it would not have any practical impact on the overall fermentation. The UV light was emitted below the critical angle of the sapphire and the solution in the bioreactor. Use of the UV light showed that it was possible to prevent biofilm formation at the crystal surface even in fermentations where biofilm was forming on the bioreactor vessel's 512a walls. The figure further shows the entrance and exit of the MIR light illuminating the MIR crystal 102a.

### Example 6—Cleaning of Probe by Adding UV Radiation

In a wastewater process plant, i.e. a slurry stream, an ATR-MIR spectroscopic analyzer 400g was used for inline monitoring of key process values like volatile fatty acid (VFA), total organic carbon (TOC), and chemical oxygen demand (COD). A MIR beam guiding probe 702a was used, comprising a stainless steel rod probe with a diamond (MIR crystal) being illuminated by the MIR light illuminating the MIR crystal 102c through mirrors 116d. The MIR beam guiding probe 702a could be inserted into the slurry. The chemometric models used in combination with the ATR-MIR spectroscopic analyzer 400g worked very well when tested in the laboratory. However, inline measurements from the process slurry became unstable after some days of inline operation, shown values obviously too high, especially for TOC and COD.

An example of this deviation over time is shown for the COD values in table 1.

| | COD g/L | COD g/L | |
|---|---|---|---|
| time (hours) | (ATR-MIR) | laboratory assay | UV LED |
| 0 | 5.20 | 5.32 | Off |
| 4 | 5.06 | 5.01 | Off |
| 8 | 5.20 | 5.23 | Off |
| 16 | 5.48 | 5.28 | Off |
| 24 | 5.81 | 5.21 | Off |
| 36 | 7.17 | 5.16 | Off |
| 42 | 9.05 | 5.24 | Off |
| 48 | 10.41 | 5.29 | Off |

Table 1 shows COD values from a wastewater stream analyzed inline using ATR-MIR and values of samples taken at the same time and analyzed with a laboratory assay method, with the UV LED turned off. Before t=0 the probe was cleaned manually

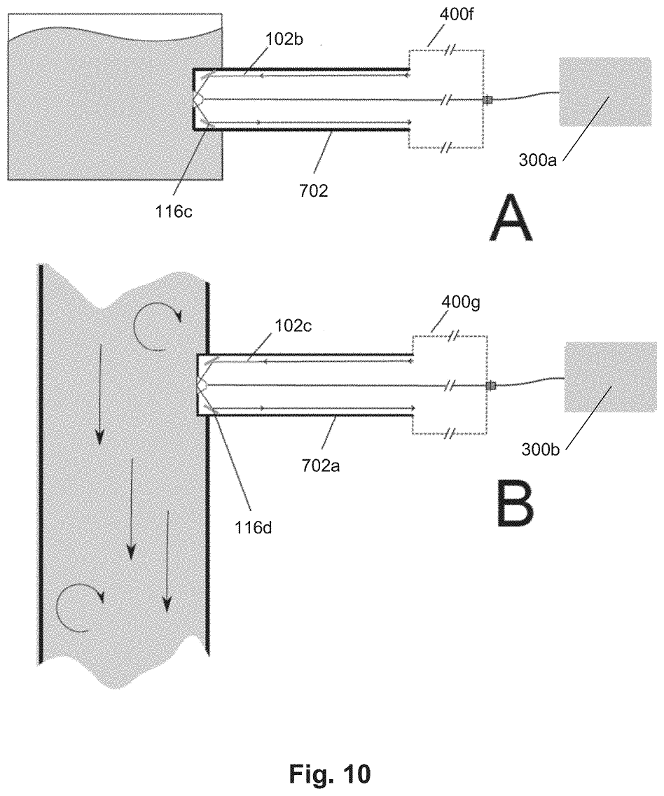

The fault in the chemometric prediction was confirmed by reference laboratory measurements. The reason was shown to be caused by the occurrence of a strong signal, especially in the 900-1200 cm⁻¹ region similar to signals associated with carbohydrates. Further strong peaks at around 1660 and 1580 cm⁻¹ was detected, indicating polyamides/proteins. The MIR beam guiding probe 702a was removed from the slurry, and rinsed with water. A slimy layer on the MIR beam guiding probe 702a would still remain after rinsing and the carbohydrate like signal would still be present. By cleaning manually with a cellulose napkin and water the MIR beam guiding probe 702a could be cleaned. Each time the MIR beam guiding probe 702a was cleaned manually and reinserted into the slurry flow the unwanted carbohydrate signal would build up again over a few days of operation. Finally, the ATR-MIR spectroscopic analyzer 400g and the MIR beam guiding probe 702a was modified with a small quartz fiber directly from the bottom of the diamond to a fiber connecter on the outside of the spectrometer. A 280 nm UV radiation source 300b (UV LED) was connected to the fiber connector at the ATR-MIR spectroscopic analyzer 400g via another quarts fiber. This allowed a portion of the 280 nm UV light to be transmitted through the diamond and into the sample. The setup used was similar to schematics shown in FIG. 10B. The ATR-MIR spectroscopic analyzer 400g and the MIR beam guiding probe 702a were reinstalled and only operated with the UV LED turned on. In this mode of operation the strong and unwanted signal from the slimy carbohydrate layer was prevented, permanently (see table 2).

| | COD g/L | COD g/L | |
|---|---|---|---|
| time(hours) | (ATR-MIR) | laboratory assay | UV LED |
| 0 | 5.19 | 5.24 | on |
| 4 | 5.10 | 5.07 | on |
| 8 | 5.18 | 5.10 | on |
| 16 | 5.36 | 5.41 | on |
| 24 | 5.63 | 5.63 | on |
| 36 | 5.34 | 5.26 | on |
| 42 | 5.21 | 5.09 | on |
| 48 | 5.18 | 5.25 | on |
| 72 | 5.07 | 5.04 | On |

Table 2 shows COD values from a wastewater stream analyzed inline using ATR-MIR and values of samples taken at the same time and analyzed with a laboratory assay method, with the UV LED turned on. Before t=0 the probe cleaned manually

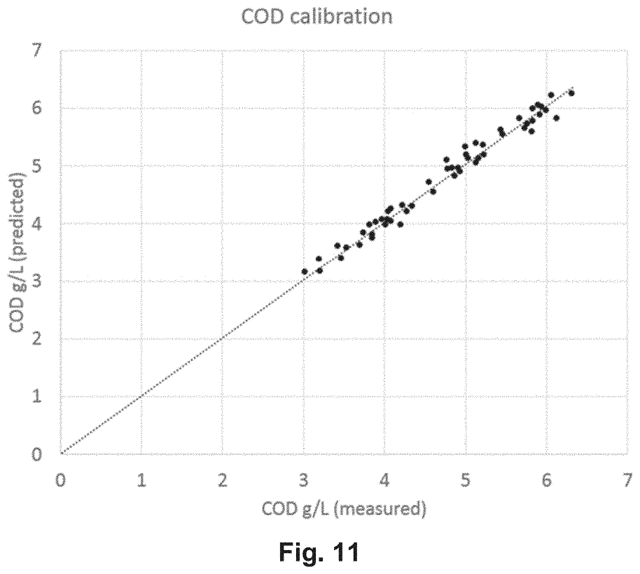

FIG. 10A shows a similar setup as FIG. 10B, and have similar results as shown above, but it is applied to a bioreactor vessel or monitoring. A plot from the calibration of the COD calibration is shown in FIG. 11, which is a plot showing PLS calibration of inline ATR-MIR prediction of COD plotted against the values found with a reference assay method.

### Example 7—Cleaning of Probe by Adding UV Radiation

In a plant producing ethanol from finely milled corn flour, an ATR-MIR spectroscopic analyzer was applied in the simultaneous saccharification and co-fermentation process (SSF process) tank, where liquefied corn slurry is converted into ethanol by glucoamylase enzyme and yeast in a large fermenter tank. A powerful positive displacement pump would create a continuous sample loop of slurry from the bottom of the tank through one inch silicon hoses. The analyzer would continuously collect MIR spectra of the slurry using a custom-made flow cell and diamond-ATR-FTIR spectrometer. A small hole was made under the mechanic support mount for the ATR diamond, where the end of a small quartz fiber was anchored, while the other end was attached to a fiber connector on the outside of the ATR optics box. A fiber on the outside of the ATR optics was connecting this to a UV LED through another quartz fiber. This allowed a germicidal dosage of UV light to reach the sample side of the diamond and be transmitted out in the slurry in close proximity to the diamond. By turning on the UV LED periodically during operation, the diamond surface could be kept clean of any biofilm formation, and ensure undisturbed operation of the Analyzer. After some optimizations it was found that the UV light did not have to be turned on at very long intervals. Finally it was found that short pules of around 30 seconds every hour were still prevent film formation. It is the combination of the length of the pulse and the interval between pulses which may optimize the film prevention. If the pulse length is shortened, the interval may also be shortened and vice versa.

### REFERENCES

100 ATR-MIR crystal
100a ATR-MIR crystal
100b ATR-MIR crystal
100c ATR-MIR crystal
102 MIR light illuminating the MIR crystal
102a MIR light illuminating the MIR crystal
102b MIR light illuminating the MIR crystal
102c MIR light illuminating the MIR crystal
104 Evanescent wave
106 MIR light reflected off the MIR crystal
108 Sample surface side of the ATR-MIR crystal
110 MIR light surface side of the ATR-MIR crystal
112 MIR emitter
114 ATR plate
116a, 116b Mirror
116c Mirror
116d Mirror
118b, 118b Other optics
120 MIR detector
124 Fiber
200 Sample
202 Microorganisms
204 Extracellular polymeric substance (EPS) matrix
206 Biofilm
208 Fluid flow over the ATR-MIR crystal
300 UV radiation source
300a UV radiation source
300b UV radiation source
302 UV radiation beam
400a ATR-MIR spectroscopic analyzer
400b ATR-MIR spectroscopic analyzer
400c ATR-MIR spectroscopic analyzer
400d ATR-MIR spectroscopic analyzer
400e ATR-MIR spectroscopic analyzer
400f ATR-MIR spectroscopic analyzer
400g ATR-MIR spectroscopic analyzer
500 UV LED
500a UV LED
502 UV Spectrometer
504 Fiber coupler
506 Quartz fiber
508 Quartz U-bend probe
510 Detector
512 Bioreactor vessel
512a Bioreactor vessel
600 Quartz lens
702 MIR beam guiding probe
702a MIR beam guiding probe

We claim:

1. An ATR-MIR (attenuated total reflectance mid-infra-red) crystal antifouling method for cleaning an ATR-MIR crystal so that biofouling on the ATR-MIR crystal is avoided, the ATR-MIR crystal being positioned in an ATR-MIR unit configured to be used for measuring ATR-MIR spectra of a sample, wherein the ATR-MIR crystal comprises:
a sample surface side in direct contact with the sample, and
an MIR light surface side onto which MIR light is directed allowing the MIR light to penetrate through the ATR-MIR crystal and interact with the sample through an evanescent wave, where the MIR light, after interacting with the sample, is reflected from the MIR surface side of the ATR-MIR crystal,
wherein the ATR-MIR crystal antifouling method comprises a step of illuminating the sample surface side of the ATR-MIR crystal with radiation from an ultra violet (UV) radiation source emitting ultra-violet (UV) light, whereby bacteria in samples in contact with the ATR-MIR crystal are killed; and
wherein UV radiation is directed at the ATR-MIR crystal from the MIR light surface side, thereby passing through the ATR-MIR crystal before illuminating the sample surface side of the ATR-MIR crystal.

2. The ATR-MIR crystal antifouling method according to claim 1, wherein the UV radiation source is placed under the ATR-crystal with the direction of a UV radiation beam lower than a critical angle of the ATR-MIR crystal.

3. The ATR-MIR crystal antifouling method according to claim 1, further comprising steps of continuously measuring ATR-MIR spectra of the sample in real time at wavelengths between 400-3500 cm-1 and directing UV radiation at the ATR-MIR crystal, said steps being executed during a time selected from:
at least part of the time during a measurement of the ATR-MIR spectra, or
all a time during a measurement of the ATR-MIR spectra, or
in between measurements of some of the ATR-MIR spectra, or
in between measurements of all of the ATR-MIR spectra.

4. The ATR-MIR crystal antifouling method according to claim 1, wherein the UV radiation source is selected from a group consisting of:
an UVC generating lamp,
a mercury lamp,
a deuterium lamp, and
a cold cathode lamp.

5. The ATR-MIR crystal antifouling method according to claim 1, wherein the UV radiation source comprises a broad spectrum lamp emitting a very high percentage of light from the UVC spectral range between 100-300 nm.

6. The ATR-MIR crystal antifouling method according to claim 1, wherein the UV radiation beam has a shape matching a shape of the sample surface side of the ATR-MIR crystal.

7. The ATR-MIR crystal antifouling method according to claim 1, wherein the sample is an aqueous slurry or is a solution comprising naturally occurring carbohydrates and proteins.

8. The ATR-MIR crystal antifouling method according to claim 1, wherein the ATR-MIR unit measures ATR-MIR spectra of an enzymatic conversion process or microbial conversion process.

9. The ATR-MIR crystal antifouling method according to claim 7, wherein the sample is a solution comprising naturally occurring carbohydrates and proteins, and said solution comprises on or more of sucrose, starch, barley, rye, wheat, oat, corn, rice, potato, straw, wood, corn stover, sugar cane, and bagasse.

10. The ATR-MIR crystal antifouling method according to claim 8, wherein a selected one of the enzymatic conversion process or microbial conversion process includes a mashing process before an ethanol fermentation process.
